(19) United States
(12) Patent Application Publication
(10) Pub. No.: US 2023/0018668 A1
(43) Pub. Date: Jan. 19, 2023

(54) HEMODIALYSIS TREATMENT MODIFICATION

(71) Applicant: Bellco S.R.L., Mirandola (IT)

(72) Inventor: Michele Varasani, SantAgata Bolognese (IT)

(21) Appl. No.: 17/812,518

(22) Filed: Jul. 14, 2022

Related U.S. Application Data
(60) Provisional application No. 63/221,749, filed on Jul. 14, 2021.

(51) Int. Cl. A61M 1/16; G16H 20/40; G16H 40/67

(52) U.S. Cl. CPC A61M 1/1603; G16H 20/40; G16H 40/67; A61M 2205/502; A61M 2205/3303

(57) ABSTRACT

An example medical device includes a sensor configured to sense a parameter of interest that changes as a function of a hemodialysis treatment parameter and generate a signal indicative of the sensed parameter of interest. The medical device includes memory configured to store an association between the parameter of interest and the hemodialysis treatment parameter. The medical device includes processing circuitry configured to receive the signal from the sensor. The processing circuitry also is configured to determine a modification to the hemodialysis treatment parameter based on the signal indicative of the parameter of interest and the association. The processing circuitry is also configured to automatically modify the hemodialysis treatment parameter based on the determined modification.

This application claims the benefit of U.S. Provisional Application No. 63/221,749, filed Jul. 14, 2021, the entire contents of which are hereby incorporated by reference herein.

### TECHNICAL FIELD

This disclosure relates to hemodialysis.

### BACKGROUND

Patients with renal disease may receive hemodialysis treatment, which may involve using a medical device, such as a dialyzer or an artificial kidney, to remove blood from the body, filter waste and extra fluid from the blood, balance electrolytes, and return the blood to the body.

### SUMMARY

The present disclosure describes devices, systems, and techniques for automatically modifying one or more hemodialysis treatment parameters based on one or more sensed parameters of interest related to the hemodialysis treatment.

To improve the efficacy of hemodialysis treatment, a medical device, such as a hemodialysis device, may store associations between one or more parameters of interest of a patient and one or more hemodialysis treatment parameters and rules regarding the application of the hemodialysis treatment parameters in memory. These associations and rules may be entered by a physician or other clinician into the medical device via a user interface of the medical device or another device. The medical device may include, or be communicatively coupled to, one or more sensors, which are configured to sense the one or more parameters of interest of a patient when the patient is receiving hemodialysis treatment. When certain parameters of interest indicate that changes to the hemodialysis treatment parameters would be beneficial to the outcome of the hemodialysis, the medical device may determine which hemodialysis treatment parameters are associated with the sensed parameters of interest, for example by reading the associations from memory, and may automatically change the hemodialysis treatment parameters based on the associations and accompanying rules. In this manner, hemodialysis treatment may be patient-specific, with input from a physician, even when the physician is not present with the patient. Additionally, as the physician or other clinician is actually entering the associations and rules for a particular patient, regulatory approvals may be more readily achieved than with a pure biofeedback system.

Aspects of this disclosure are directed to techniques for changing treatment parameters for a patient. In some examples, a medical device includes a sensor configured to sense a parameter of interest that changes as a function of a hemodialysis treatment parameter and generate a signal indicative of the sensed parameter of interest; memory configured to store an association between the parameter of interest and the hemodialysis treatment parameter; and processing circuitry configured to: receive the signal from the sensor; determine a modification to the hemodialysis treatment parameter based on the signal indicative of the parameter of interest and the association; and automatically modify the hemodialysis treatment parameter based on the determined modification.

In some examples, a method includes receiving, by processing circuitry, a signal indicative of a sensed parameter of interest from a sensor; determining, by the processing circuitry, a modification to a hemodialysis treatment parameter based on the signal indicative of the parameter of interest and an association, stored in memory, between the parameter of interest and a hemodialysis treatment parameter; and automatically modifying, by the processing circuitry, the hemodialysis treatment parameter based on the determined modification.

In some examples, a non-transitory computer-readable storage medium stores instructions that, when executed, cause processing circuitry to: receive a signal indicative of a sensed parameter of interest from a sensor; determine a modification to a hemodialysis treatment parameter based on the signal indicative of the parameter of interest and an association, stored in memory, between the parameter of interest and a hemodialysis treatment parameter; and automatically modify the hemodialysis treatment parameter based on the determined modification.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram of an example medical device and a patient.

FIG. 2 is a functional block diagram illustrating an example configuration of an example medical device.

FIG. 3 is a flowchart illustrating an exemplary operation of a medical device in accordance with the techniques of this disclosure.

Like reference characters denote like elements throughout the description and figures.

### DETAILED DESCRIPTION

While receiving hemodialysis treatment, parameters of interest of the patient may be monitored via one or more sensors. For example, vital signs, such as blood pressure, pulse, and temperature of the patient, may be monitored via external sensors to determine whether the hemodialysis treatment is being well-received by the patient. Additionally, one or more other parameters of interest may be monitored via one or more sensors within the hemodialysis device itself, such as blood volume, blood oxygen saturation, blood temperature, and the like.

Different patients may have different states of disease and different comorbidities. As such, an efficacious hemodialysis treatment program including hemodialysis treatment parameters may vary from patient to patient. Additionally, a given treatment program may not always be well-received by the same patient, as the state of health of the patient may change over time.

During hemodialysis treatment, a physician may not always be present with the patient. When certain indications of parameters of interest are received from one or more sensors monitoring the patient, a change in the hemodialysis treatment parameters may be needed to improve the efficacy of the hemodialysis treatment, to preserve the health of the patient, or to investigate potential issues with the hemodialysis treatment. While other medical personnel may be present with the patient if the patient is receiving hemodialysis treatment at a clinical facility, the medical personnel may not be best suited for changing the treatment parameters, as each patient may respond differently to different hemodialysis treatment parameters. It may be preferable to have any change in hemodialysis treatment parameters be driven by a physician.

Biofeedback systems may be incorporated into a hemodialysis device. However, the use of a pure biofeedback system (e.g., a biofeedback system without physician oversight) in a medical device may require lengthy and difficult regulatory approvals, as different patients may react differently to any treatment parameters that a pure biofeedback system may determine.

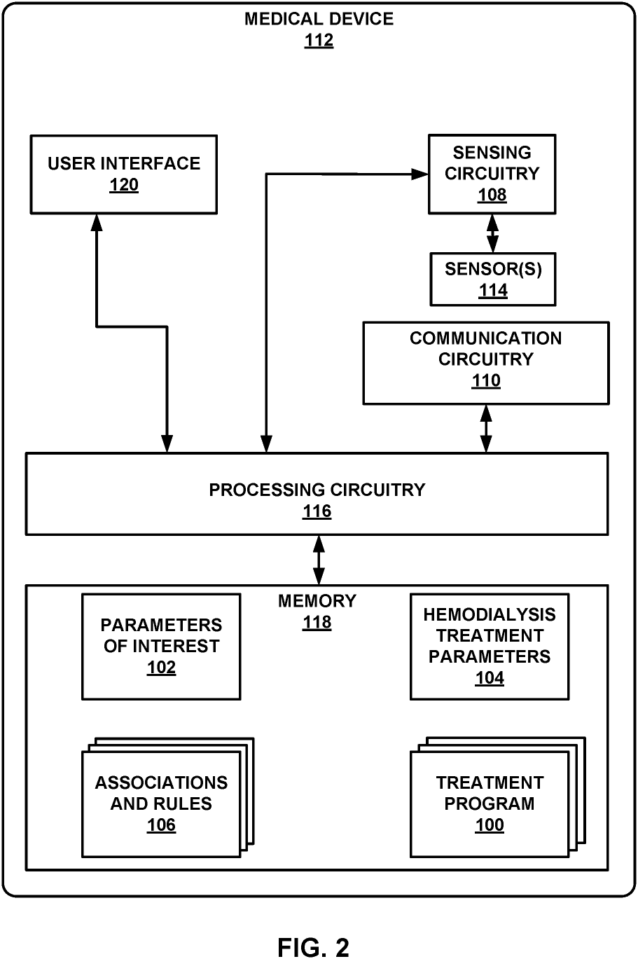

This disclosure describes a medical device, such as a hemodialysis device, that is configured to determine that sensed parameters of interest indicate that a change in hemodialysis treatment parameters should be made (e.g., to improve efficacy or to maintain a desired patient health state) and to automatically make those changes in hemodialysis treatment parameters based on stored associations between the parameters of interest and the hemodialysis treatment parameters, such that the medical device provides treatment based on the change in the hemodialysis treatment parameters. In some examples, the medical device may include one or more sensors and/or be communicatively coupled to one or more sensors which may be external to the medical device. The one or more sensors are configured to sense one or more parameters of interest and to generate one or more signals indicative of the sensed one or more parameters of interest. For example, the parameters of interest may include blood pressure (e.g., diastolic pressure, systolic pressure, and/or medium pressure), heart rate, blood volume change (e.g., percentage of change), and the like. A more detailed list of example parameters of interest is provided below with respect to the discussion of FIG. 2.

The medical device may provide treatment to a patient based on one or more hemodialysis treatment parameters. For example, the hemodialysis treatment parameters may include sodium concentration of a dialysis solution, an ultrafiltration rate, an ultrafiltration volume, a duration of a hemodialysis session, temperature of filtration, and the like. A more detailed list of example treatment parameters is provided below with respect to the discussion of FIG. 2.

The medical device may include memory that stores associations between the one or more parameters of interest and the one or more hemodialysis treatment parameters and rules for changing the one or more treatment parameters based on associated parameter(s) of interest. For example, any of the parameters of interest may be associated with any of the hemodialysis treatment parameters. In some examples, a plurality of parameters of interest may be associated with a single hemodialysis treatment parameter. In some examples, a single parameter of interest may be associated with a plurality of hemodialysis treatment parameters. In some examples, a plurality of parameters of interest may be associated with a plurality of hemodialysis treatment parameters. For example, the stored associations may be used, by the hemodialysis device, for example, to automatically change any of the hemodialysis treatment parameters based on one or more events occurring involving any of the parameters of interest. Examples of associations and changes in hemodialysis treatment parameters based on parameters of interest are listed below with respect to the discussion of FIG. 2.

For example, there may be investigatory or corrective actions taken based on events occurring involving any of the parameters of interest. Investigatory actions may be used in the case of suspected limited performance of hemodialysis treatment session or risk of adverse impact to the patient so as to provide a clinician with more information that may be used to better understand the performance or risk. Corrective actions may include adjusting settings of the hemodialysis treatment session to improve the efficacy of a hemodialysis treatment session.

The medical device may also include processing circuitry configured to automatically (e.g., without user intervention) change one or more hemodialysis treatment parameters based on the sensed one or more parameters of interest and the association between the one or more parameters of interest and the one or more hemodialysis treatment parameters. While the medical device and techniques of this disclosure are primarily discussed as being a hemodialysis device and hemodialysis techniques, the medical device and techniques may be any other type of medical device or techniques which may benefit from the techniques of this disclosure.

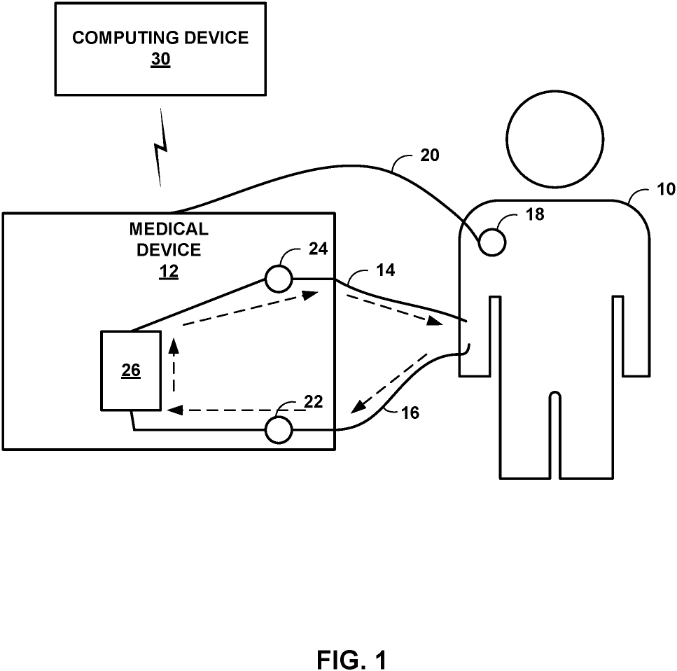

FIG. 1 is a conceptual diagram of an example hemodialysis system and patient 10. The hemodialysis system includes a medical device 12 and computing device 30. The dashed arrows indicate the direction of blood flow during a hemodialysis session.

A clinician (or other user) may fluidically connect arterial line 16 to an inflow port (not shown) on medical device 12 and to an intravenous catheter (e.g., to a first lumen of the catheter), an arteriovenous fistula, or a synthetic graft (not shown) in patient 10 to provide access to the vasculature of patient 10. The arteriovenous fistula or the synthetic graft in the patient may be accessed, for example, via a needle or cannula. Arterial line 16 may be configured to facilitate the transport of blood from an artery of patient 10 to medical device 12. For example, blood from patient 10 may contain high levels of waste products due to kidney failure or kidney disease.

The clinician may also fluidically connect a venous line 14 to an outflow port (not shown) of the medical device 12 and to the intravenous catheter (e.g., to a second lumen of the catheter), the arteriovenous fistula or the synthetic graft in patient 10 to provide access to a vein of patient 10. Venous line 14 may be configured to return relatively cleaner blood from medical device 12 to the vasculature of patient 10.

Medical device 12 is configured to remove waste products from the blood received via arterial line 16. For example, medical device 12 may include a dialyzer 26 and/or one or more filters that may remove waste products and excess fluid from the blood received via arterial line 16. Dialyzer 26 may use a dialysate solution to remove the waste products and excess fluid from the blood of patient 10. Medical device 12 includes one or more sensors, such as sensor 22 and/or sensor 24. For example, sensor 22 or sensor 24 may be an arterial pressure sensor, a venous pressure sensor, an air sensor, an inflow pressure sensor (e.g., a sensor that senses the pressure of the inflow into dialyzer 26), a blood temperature sensor, a hematocrit sensor, a conductivity sensor, an optical sensor, a chemical sensor (for sensing levels of glucose or other substances), or the like. While depicted as part of medical device 12, in some examples, sensor 22 and/or sensor 24 may be external to medical device 12, for example, in arterial line 16 or venous line 14.

In some examples, medical device 12 may be communicatively coupled to one or more external sensors, such as one or more sensors 18, via a link 20. Link 20 may be a wired link, a wireless link, an optical link, or any combination thereof. One or more sensors 18 may be configured to sense physiological parameters of interest of patient 10 and generate one or more signals indicative of the sensed one or more parameters of interest. For example, one or more sensors 18 may sense one or more of blood pressure, temperature, oxygen content of the blood, heart rate, weight, bioimpedance, bleeding (for example, close to a vascular access connection, such as a fistula), removal of urea or different molecules from blood, and the like. Sensor 22 may be configured to sense a parameter of interest in blood entering medical device 12 and sensor 24 may be configured to sense a parameter of interest in blood exiting medical device 12. Medical device 12 may also include a blood pump (not shown) which is configured to keep the blood of patient 10 flowing through medical device 12.

In some examples, medical device 12 may be communicatively coupled to computing device 30. In some examples, computing device 30 may be a dedicated external programmer used to program medical device 12. In some examples, computing device 30 may be an off the shelf device, such as a smart phone, a tablet, a laptop computer, a desktop computer, or the like, and be configured to program medical device 12. Computing device 30 may include communication circuitry to communicatively connect to medical device 12 via a wireless, wired, or optical connection.

FIG. 2 is a functional block diagram illustrating an example configuration of an example medical device. Medical device 112 may be an example of medical device 12 of FIG. 1. In some examples, computing device 30 may be similar to medical device 112, such as include one or more components of medical device 112. While a number of components of the medical device 112 are shown, certain components are not shown. For example, in the case where medical device 112 is a hemodialysis device, medical device 112 may include a blood pump, a dialyzer, one or more filters, and the other components which may be used to provide dialysis treatment to patient 10 (FIG. 1).

In the example of FIG. 2, medical device 112 includes processing circuitry 116, user interface 120, sensing circuitry 108, sensors 114, communication circuitry 110, and memory 118. Processing circuitry 116 may include any of a microprocessor, integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 116 may include multiple components, such as any combination of one or more microprocessors, one or more DSPs, one or more ASICs, or one or more FPGAs, as well as other discrete or integrated logic circuitry, and/or analog circuitry.

Memory 118 may store program instructions which may include one or more program modules, which are executable by processing circuitry 116. When executed by processing circuitry 116, such program instructions may cause processing circuitry 116 to provide the functionality ascribed to processing circuitry 116 described herein. The program instructions may be embodied in software and/or firmware. Memory 118 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Communication circuitry 110 may be configured to communicate with one or more sensors 18 or computing device 30 (both of FIG. 1) via wired, optical, or wireless communication. In some examples, computing device 30 may communicate via near field communication (NFC) technologies (e.g., inductive coupling, NFC or other communication technologies operable at ranges less than 10-20 cm) and/or far field communication technologies (e.g., Radio Frequency (RF) telemetry according to the 802.11, Bluetooth® specification sets, or other communication technologies operable at ranges greater than NFC technologies.

In some examples, medical device 112 stores in memory 118 a treatment program 100 for patient 10. If medical device 112 is used to treat more than one patient, medical device 112 may store multiple treatment programs 100 such that each patient may have an associated personalized treatment program 100. Treatment program 100 may include various hemodialysis treatment parameters that may be used when treating a patient, such as patient 10.

Medical device 112 may also store in memory 118 parameters of interest 102. Parameters of interest 102 may include one or more parameters that may be sensed by sensors 114 and/or by external sensors (e.g., one or more sensors 18) communicatively coupled to medical device 112, such as one or more sensors 18 of FIG. 1. For example, parameters of interest may include blood pressure (e.g., diastolic pressure, systolic pressure, and/or medium pressure), heart rate, blood volume change (e.g., percentage of change), blood oxygen saturation, blood temperature (e.g., in venous line 14, arterial line 16, in a body of patient 12, or the like) aspiration pressure in arterial line 16 (e.g., prior to the blood pump), inlet pressure in arterial line 16 (e.g., before dialyzer 26, but after the blood pump), pressure in venous line 14 (e.g., prior to entering vasculature of patient 12), one or more infusion pressures (e.g., in the context of hemodiafiltration or hemofiltration), filter inlet pressure to a dialysate compartment of medical device 112, filter outlet pressure from the dialysate compartment, transmembrane pressure (e.g., which may be calculated as transmembrane pressure=[(blood pressure in + blood pressure out)/2 - (filter inlet pressure in the dialysate compartment + filter outlet pressure in the dialysate compartment)/2] or maximum transmembrane pressure=(blood pressure in -filter outlet pressure in the dialysate compartment) or minimum transmembrane pressure=(blood pressure out-filter inlet pressure in the dialysate compartment) or any similar value of transmembrane pressure, hematocrit in arterial line 16, hematocrit in venous line 14, hemoglobin (e.g., g/dl) in arterial line 16, hemoglobin in venous line 14, oxygen saturation in arterial line 16, oxygen saturation in venous line 14, glucose concentration in the blood, recirculation of the vascular access, dialysis adequacy (e.g., Kt/V, where K is dialyzer clearance or the rate at which blood passes through the dialyzer (e.g., milliliters per minute), t is time, and V is volume of water in the body of a patient), other possible parameters indicative of dialysis dose or dialysis efficiency (e.g., dialysance method or optical absorbance method), or the like. While several example parameters of interest have been disclosed, other parameters of interest may be sensed according to the techniques of this disclosure.

In some examples, medical device 112 also stores in memory 118 hemodialysis treatment parameters 104. Hemodialysis treatment parameters 104 may include one or more dialysis settings used for hemodialysis treatment of patient 10 by medical device 112 or another medical device. In some examples, any of hemodialysis treatment parameters 104 may be part of treatment program 100. For example, hemodialysis treatment parameters 104 may include sodium concentration (or total conductivity) of a dialysate solution, an ultrafiltration rate, a total ultrafiltration volume (e.g., patient weight loss set for the hemodialysis session), a duration of a hemodialysis session, temperature of filtration, sodium concentration of the dialysate, blood flow rate (QB) implemented by medical device 112 or another medical device during a dialysis session, dialysate flow rate (Qd), duration of dialysis, dialysate temperature, bicarbonate concentration (or partial conductivity) of the dialysate solution, concentrate composition (e.g., when 2 or more different acid concentrates are used to provide hemodialysis treatment to patient 12 by medical device 112 or another medical device), total ultrafiltration volume, infusion rate in case of hemodiafiltration (HDF) and hemofiltration (HF) techniques, bolus volume, systemic blood pressure measurement, clearance measurement (e.g., by ionic dialysance techniques or equivalent techniques), vascular access recirculation measurement, and the like. While several example hemodialysis treatment parameters have been disclosed, other hemodialysis treatment parameters may be used to provide treatment and/or investigate treatment according to the techniques of this disclosure.

In some examples, a clinician can provide input indicative of treatment program 100 via user interface 120. User interface 120 may include a display, touch screen, keypad, mouse, microphone, speaker, or other device that may facilitate the interaction of a user, such as a clinician, and medical device 112. In some examples, the clinician may enter treatment program 100 on computing device 30 and computing device 30 may communication treatment program 100 to medical device 12 via a communications link therebetween.

In some examples, a clinician can access parameters of interest 102 and hemodialysis treatment parameters 104 via user interface 120. In some examples, user interface 120 may display a selectable list of parameters of interest and a selectable list of hemodialysis treatment parameters. User interface 120 may facilitate clinician creating or editing associations between any of parameters of interest 102 and any of hemodialysis treatment parameters 104. In some examples, user interface 120 may also facilitate a clinician creating or editing rules that accompany the associations. For example, a clinician may use user interface 120 to program a sequence of one or more actions based on one or more of parameters of interest 102 that may change one or more of hemodialysis treatment parameters 104 being used for treatment (e.g., associations and rules 106). Medical device 112 may provide hemodialysis treatment to patient 10 based on the change in the one or more hemodialysis treatment parameters.

In some examples, rather than accessing parameters of interest 102 and hemodialysis treatment parameters 104 via user interface 120, a clinician may access parameters of interest 102 and hemodialysis treatment parameters 104 on computing device 30 (either through a communications link between computing device 30 and medical device 12 or via local memory on computing device 30). In such an example, the clinician may program associations and rules 106 on computing device 30 and computing device 30 may communication associations and rules 106 to medical device 12 via the communications link.

A clinician may use user interface 120 to program association and rules 106, which may be stored in memory 118. For example, processing circuitry 116 may use association and rules 106 to automatically change hemodialysis treatment parameters used in treating patient 10 based on one or more sensed parameters of interest. For example, rules of associations and rules 106 can specify specific events (e.g., as indicated by a sensed parameter of interest crossing a respective threshold value or falling outside a predetermined range of values) for, or a combination of events for a plurality of parameters of interest. As an example, one of associations and rules 106 may indicate that in if one or more sensors 18 or sensors 114 sense that x parameter of interest changed by y amount for z minutes, then change hemodialysis treatment parameter a to treatment parameter b. Processing circuitry 116 of medical device 112 may then monitor the sensed parameters of interest and when x parameter of interest changed by y amount for z minutes, processing circuitry 116 may automatically change the hemodialysis treatment parameter a to treatment parameter b.

Memory 118 stores associations between the one or more parameters of interest and the one or more hemodialysis treatment parameters. For example, any of the parameters of interest may be associated with any of the hemodialysis treatment parameters. In some examples, a plurality of parameters of interest may be associated with a single hemodialysis treatment parameter. In some examples, a single parameter of interest may be associated with a plurality of hemodialysis treatment parameters. In some examples, a plurality of parameters of interest may be associated with a plurality of hemodialysis treatment parameters. For example, the stored associations may be used, by the hemodialysis device, for example, to automatically change any of the hemodialysis treatment parameters based on one or more events occurring involving any of the parameters of interest.

For example, processing circuitry 116 make take a predetermined investigatory or corrective action based on events occurring involving any of the parameters of interest. Investigatory actions may be used in the case of suspected limited performance of a hemodialysis treatment session or potential adverse impact to the patient so as to provide a clinician with more information to better understand the performance or risk. Corrective actions may include adjusting hemodialysis settings to improve the efficacy of a hemodialysis treatment session.

An example of an investigatory action is measuring of systemic blood pressure in response to determining a reduction in blood volume reaches a predetermined threshold value, and/or detection of a threshold change of in oxygen saturation of blood (e.g., an absolute reduction below a programmable threshold or a floating of the mean value), and/or an increase of heart rate over a predetermined threshold or a relative change over a predetermined threshold.

Another example of an investigatory action is measuring of clearance (by dialysance techniques or equivalent techniques) in response to an increase of transmembrane pressure (TMP) over a programmable threshold as a partial clotting of the dialyzer could reduce efficacy of hemodialysis treatment.

Another example of an investigatory action is measuring vascular access recirculation in response to detecting, based on the sensed parameters of interest, a reduction of K - Kt - Kt/V (which may be due to recirculation), and/or an increase of hematocrit (or a reduction of blood volume percentage) over a predetermined threshold. For example, if the blood volume reduction is not compatible with the programmed ultrafiltration rate and the body size of the patient, then recirculation may be the cause of a detected reduction of K - Kt - Kt/V or the increase of hematocrit.

An example of a corrective action is adjusting blood flow rate from patient 12 into medical device 112 to improve the hemodialysis treatment in response to detected low aspiration pressure (e.g., aspiration pressure is less than or equal to a predetermined threshold), and/or low pressure in venous line 14 (e.g., the pressure in venous line 14 is less than or equal to a predetermined threshold), and/or no high TMP (e.g., TMP being above or below a configurable threshold), and/or K - Kt - Kt/V falling below a predetermined threshold.

Another example of a corrective action is adjusting the dialysate flow rate (Qd) to improve the hemodialysis treatment in response to blood flow not being adequate (e.g., the ratio of blood flow/dialysate flow is outside of a programmable range), and/or K - Kt - Kt/V falling below a predetermined threshold.

Another example of a corrective action is adjusting the dialysate temperature in response to a blood temperature not being within an expected predetermined temperature range, and/or reduction of systemic blood pressure of the patient beyond a predetermined threshold, and/or increase of patient heart rate to be greater than or equal to a predetermined threshold value.

Another example of a corrective action is adjusting the sodium concentration (or total conductivity) of the dialysis solution in response to a reduction of blood volume percentage being less than or equal to a predetermined threshold, and/or a predetermined change of systemic blood pressure of the patient (e.g., a blood pressure increase or decrease that is more than a programmable threshold).

Another example of a corrective action is adjusting the bicarbonate concentration (or partial conductivity) in the dialysis solution in response to changes in sodium concentration relative to a predetermined sodium concentration range or value.

Another example of a corrective action, if the hemodialysis device is connected to more than one centralized distribution ring, is changing the acid concentrate in response to a significant change of systemic blood pressure (e.g., a blood pressure increase or decrease that is more than a programmable threshold), and/or a significant change in heart rate (e.g., a heart rate increase or decrease that is more than a programmable threshold).

Another example of a corrective action is changing the total duration of a hemodialysis treatment session in response to an expected targeted total ultrafiltration not being reachable because the ultrafiltration rate should be reduced, and/or the targeted Kt -Kt/V is not reachable. For example, processing circuitry 116 may extend the duration of a hemodialysis treatment session if Kt is too low, or reduce the duration of treatment if the targeted Kt is achieved prior to the scheduled end of treatment.

Another example of a corrective action is adjusting the total ultrafiltration volume in response to repeated reduction of systemic blood pressure, and/or a reduction of the dialysis session duration, and/or a reduction of blood volume percentage below a programable threshold.

Another example of a corrective action is adjusting the ultrafiltration rate in response to a reduction of systemic blood pressure below a predetermined threshold, and/or reduction of blood volume percentage below a predetermined threshold, and/or when there is no possibility to extend the duration of the hemodialysis treatment session.

Another example of a corrective action is adjusting the infusion rate in response to changes of the expected infusion volume target, and/or changes of the duration of dialysis session, and/or changes of the blood flow, and/or changes of the TMP (or any other pressure detected in the extracorporeal blood circuit), and/or changes of systemic blood pressure, and/or changes of the blood volume percentage, and/or changes in oxygen saturation.

Another example of a corrective action is adjusting the infusion modality in HDF or HF in response to changes of TMP (and any other pressure in extracorporeal blood circuit), and/or changes of hematocrit, and/or changes of blood flow.

Another example of a corrective action is infusing of a bolus of a certain volume into vasculature of patient 12 in response to an increase of hematocrit greater than a predetermined threshold, and/or a reduction of blood volume percentage, and/or an increase of TMP (or any other pressure in the extracorporeal circuit).

Processing circuitry 116 may automatically implement any of the above investigatory and corrective actions based on signals from sensors 114, each signal changing as a function of respective parameters of interest. While several examples of investigatory and corrective actions have been set forth, any hemodialysis treatment parameter may be adjusted or changed automatically in response to any parameter of interest. Additionally, the predetermined thresholds, predetermined ranges, or the like in associations and rules 104 may include programmable thresholds, programmable ranges, or the like, which may be programmed by a clinician and applied to any of the parameters of interest to determine if associated hemodialysis treatment parameters should be changed. The programmable thresholds or programmable ranges may include values, percentages, slopes, derivatives, or the like. The programmable thresholds or programmable ranges may be patient-specific in order to provide patient-specific dialysis treatment.

In some examples, processing circuitry 116 may continue to monitor parameters of interest and, if the associations and rules 106 do not indicate another change needs to be made to the treatment parameters, then processing circuitry 116 may update treatment program 100 to include the changed treatment parameter(s) or may provide a notification to the clinician asking the clinician if they desire to incorporate the changed treatment parameter(s) in treatment program 100.

In some examples, association and rules 106 includes conditions. For example, association and rules 106 may include a threshold or range. If a parameter of interest meets or exceeds the threshold or exits the range, the automatic change in the associated hemodialysis treatment parameter by processing circuitry 116 may be triggered. In some examples, association and rules 106 includes “ands” and “ors” that may string conditions together. For example, if parameter of interest x is greater than or equal to threshold t “and” parameter of interest y is outside of range r, then processing circuitry 116 can change hemodialysis treatment parameter a to hemodialysis treatment parameter b “or” change hemodialysis treatment parameter a to hemodialysis treatment parameter c.

In some examples, associations and rules 106 may use direct values of any sensed parameter of interest applied against a threshold or range or may combine values of more than one parameter of interest in any manner (e.g., add, subtract, multiply, divide, and the like) and apply such a combined value against a threshold value or a predetermined range of values. In some examples, associations and rules 106 may include rules relating to trends of one or more of parameters of interest 102 over time.

Treatment program 100 and association and rules 106 may be separately programmable for each patient that may be treated using medical device 112. In this manner, a clinician may adapt treatment to an individual patient, as well as provide for individualized automatic changes to treatment based on sensed parameters of interest. In some examples, associations and rules 106 may be configured to provide an alert to the clinician based on the sensed parameter of interest. In this manner, medical device 112 may employ techniques that may make use of biofeedback techniques, while allowing for personalization of associations and rules 106 by a clinician. For example, treatment program 100 may be different for a first patient than a second patient. Similarly, associations and rules 106 may be different for the first patient than the second.

For example, oxygen saturation level of blood of the patient may be detected by an optical sensor which may be one of sensors 114. A clinician may program associations and rules 106 for patient 10 such that in response to determining a standard deviation of a mean value of the sensed oxygen saturation is greater than or equal to a threshold t for more than a programmed time limit, such as 5 minutes to 20 minutes, e.g., 15 minutes, processing circuitry 116 modifies medical device 12 to modify one or more hemodialysis treatment parameters. The threshold t can be selected to, for example, indicate an occurrence of a hypotensive episode of the patient or a likely occurrence of the hypotensive episode.

In some examples, a clinician may analyze data offline (e.g., on computing device 30) to determine which changes to hemodialysis treatment parameters affect which parameters of interest and utilize such an analysis when programming associations and rules 106.

In some examples, a clinician may program associations and rules 106 such that in response to determining a sensed blood volume reduction increases by more than a predetermined percentage in a predetermined time period (e.g., 10% in 15 minutes), processing circuitry 116 reduces a concentration rate of dialysate by a predetermined percentage, such as a percentage in the range of 0.5% to 3%. As another example, a clinician may program associations and rules 106 such that in response to determining a sensed blood volume increases by more than 10% in 15 minutes, then processing circuitry 116 changes a duration or a temperature of filtration. The blood volume reduction by a predetermined percentage in a predetermined time period may indicate, for example, a hypotensive episode is occurring or is likely to occur.

In one example, a clinician may program associations and rules 106 such that an ultrafiltration rate and/or sodium concentration in a dialysis solution change if the derivate of the blood volume curve overcomes a programmable threshold, such as 5% to 20%, or 10%. This example could be programmed by a clinician in associations and rules 106 via user interface 120 and could be used as an alternative to an embedded biofeedback system while providing customized and personalized automatic changes in treatment for patient 10. The programmable threshold may be patient-specific in order to provide patient-specific dialysis treatment. In some examples, the programmable threshold is also a rate of change, e.g., a predetermined percentage a predetermined period of time, as the rate of change may be indicative of the significance of the change in blood volume, particularly as it relates to modifying a hemodialysis treatment parameter.

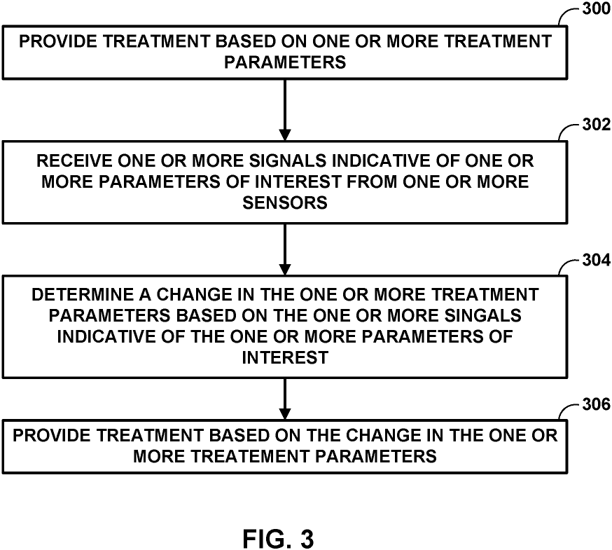

FIG. 3 is a flow diagram illustrating example techniques for changing treatment parameters of a medical device. Processing circuitry 116 may receive a signal indicative of a sensed parameter of interest from a sensor (300). For example, processing circuitry 116 may receive a signal from any of one or more sensors 18 or from any of sensor(s) 114. Such a signal may be indicative of a parameter of interest.

Processing circuitry 116 may determine a modification to a hemodialysis treatment parameter based on the signal indicative of the parameter of interest and an association, stored in memory, between the parameter of interest and a hemodialysis treatment parameter (302). For example, processing circuitry 116 may determine that the parameter of interest has changed. Processing circuitry 116 may access association and rules 106 for patient 10. Processing circuitry 116 may determine that the parameter of interest is associated with the hemodialysis treatment parameter and that one or more rules are satisfied such that a particular change in the hemodialysis treatment parameter should be made.

Processing circuitry 116 may automatically modify the hemodialysis treatment parameter based on the determined modification (304). For example, processing circuitry 116 may automatically change the hemodialysis treatment parameter as dictated by associations and rules 106 such that medical device 112 provides treatment to patient 10 according to the changed hemodialysis treatment parameter.

In some examples, medical device 112 is a hemodialysis device. In some examples, the sensor is a first sensor, the signal is a first signal, the hemodialysis treatment parameter is a first hemodialysis treatment parameter, the association is a first association, and the modification is a first modification. In such examples, processing circuitry 116 may receive a second signal indicative of a sensed second parameter of interest from a second sensor (e.g., one of sensor(s) 114 or one of one or more sensors 18 (FIG. 1)). Processing circuitry 116 may determine a second modification to a second hemodialysis treatment parameter based on the second signal indicative of the sensed second parameter of interest and a second association stored in memory 118. Processing circuitry 116 may automatically modify the second hemodialysis treatment parameter based on the determined second modification.

In some examples, the parameter of interest includes one of blood pressure, heart rate, blood volume change, blood oxygen saturation, blood temperature, heart rate, aspiration pressure in an arterial line, inlet pressure in the arterial line, pressure in a venous line, infusion pressure, filter inlet pressure to a dialysate compartment, filter outlet pressure from the dialysate compartment, transmembrane pressure, maximum transmembrane pressure, minimum transmembrane pressure minimum, hematocrit in the arterial line, hematocrit in the venous line, hemoglobin in the arterial line, hemoglobin in the venous line, oxygen saturation in the arterial line, oxygen saturation in the venous line, glucose concentration in blood, vascular access recirculation, or Kt/V, wherein K is dialyzer clearance, t is time, and V is a volume of water in the body of a patient.

In some examples, the hemodialysis treatment parameter includes one of sodium concentration of a dialysis solution, ultrafiltration rate, total ultrafiltration volume, duration of a hemodialysis session, temperature of filtration, blood flow, dialysate flow, dialysate temperature, bicarbonate concentration of the dialysate solution, concentrate composition, total ultrafiltration volume infusion rate, bolus volume, systemic blood pressure measurement, clearance measurement, or vascular access recirculation measurement.

In some examples, medical device 112 may determine that an indication of the parameter of interest is greater than or equal to a predetermined threshold for a predetermined period of time, wherein the determining the modification to the hemodialysis treatment parameter is further based on the determination that the indication of the parameter of interest is greater than or equal to the predetermined threshold for the predetermined period of time. In some examples, the predetermined period of time is in the range of 10 minutes to 20 minutes. In some examples, the indication of the parameter of interest is a standard deviation of the parameter of interest from a mean of the parameter of interest. In some examples, the indication of the parameter of interest is a percentage of increase or decrease of the parameter of interest.

In some examples, user interface 120 of medical device 112 may display a list of a plurality of parameters of interest and a list of a plurality of hemodialysis treatment parameters. User interface 120 may accept user input associating the parameter of interest with the hemodialysis treatment parameter. Processing circuitry 116 may store the association in memory 118.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

Based upon the above discussion and illustrations, it is recognized that various modifications and changes may be made to the disclosed technology in a manner that does not necessarily require strict adherence to the examples and applications illustrated and described herein. Such modifications do not depart from the true spirit and scope of various aspects of the disclosure, including aspects set forth in the claims.

In at least one example, the functions described may be implemented in hardware, software, firmware, or any combination thereof. If implemented in software, the functions may be stored on or transmitted over as at least one instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by at least one computers or at least one processors to retrieve instructions, code and/or data structures for implementation of the techniques described in this disclosure. A computer program product may include a computer-readable medium.

By way of example, and not limitation, such computer-readable data storage media can include RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Combinations of the above should also be included within the scope of computer-readable media.

Instructions may be executed by at least one processors, such as at least one DSPs, general purpose microprocessors, ASICs, FPGAs, CPLDs, or other equivalent integrated or discrete logic circuitry. Accordingly, the term “processor” or “processing circuitry” as used herein may refer to any of the foregoing structure or any other structure suitable for implementation of the techniques described herein. Also, the techniques could be fully implemented in at least one circuits or logic elements.

Any of the above-mentioned “processors,” and/or devices incorporating any of the above-mentioned processors or processing circuitry, may, in some instances, be referred to herein as, for example, “computers,” “computer devices,” “computing devices,” “hardware computing devices,” “hardware processors,” “processing units,” “processing circuitry,” etc. Computing devices of the above examples may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In some examples, the computing devices may be controlled by a proprietary operating system. Conventional operating systems control and schedule computer processes for execution, perform memory management, provide file system, networking, I/O services, and provide UI functionality, such as GUI functionality, among other things.

The techniques of this disclosure may be implemented in a wide variety of devices or apparatuses, including an integrated circuit (IC) or a set of ICs (e.g., a chip set). Various components, modules, or units are described in this disclosure to emphasize functional aspects of devices configured to perform the disclosed techniques, but do not necessarily require realization by different hardware units.

This disclosure includes the following non-limiting examples.

Example 1. A medical device comprising: a sensor configured to sense a parameter of interest that changes as a function of a hemodialysis treatment parameter and generate a signal indicative of the sensed parameter of interest; memory configured to store an association between the parameter of interest and the hemodialysis treatment parameter; and processing circuitry configured to: receive the signal from the sensor; determine a modification to the hemodialysis treatment parameter based on the signal indicative of the parameter of interest and the association; and automatically modify the hemodialysis treatment parameter based on the determined modification.

Example 2. The medical device of example 1, wherein the medical device is a hemodialysis device.

Example 3. The medical device of example 1 or example 2, wherein the sensor is a first sensor, the signal is a first signal, the hemodialysis treatment parameter is a first hemodialysis treatment parameter, the association is a first association, and the modification is a first modification, the medical device further comprising: a second sensor configured to sense a second parameter of interest that changes as a function of a second hemodialysis treatment parameter and generate a second signal indicative of the sensed second parameter of interest; wherein the memory is further configured to store a second association between the second parameter of interest and the second hemodialysis treatment parameter, and wherein the processing circuitry is further configured to: receive the second signal from the second sensor; determine a second modification to the second hemodialysis treatment parameter based on the second signal indicative of the sensed second parameter of interest and the second association; and automatically modify the second hemodialysis treatment parameter based on the determined second modification.

Example 4. The medical device of any of examples 1-3, wherein the parameter of interest comprises one of blood pressure, heart rate, blood volume change, blood oxygen saturation, blood temperature, heart rate, aspiration pressure in an arterial line, inlet pressure in the arterial line, pressure in a venous line, infusion pressure, filter inlet pressure to a dialysate compartment, filter outlet pressure from the dialysate compartment, transmembrane pressure, maximum transmembrane pressure, minimum transmembrane pressure minimum, hematocrit in the arterial line, hematocrit in the venous line, hemoglobin in the arterial line, hemoglobin in the venous line, oxygen saturation in the arterial line, oxygen saturation in the venous line, glucose concentration in blood, vascular access recirculation, or Kt/V, wherein K is dialyzer clearance, t is time, and V is a volume of water in the body of a patient.

Example 5. The medical device of any of examples 1-4, wherein the hemodialysis treatment parameter comprises one of sodium concentration of a dialysis solution, ultrafiltration rate, total ultrafiltration volume, duration of a hemodialysis session, temperature of filtration, blood flow, dialysate flow, dialysate temperature, bicarbonate concentration of the dialysate solution, concentrate composition, total ultrafiltration volume infusion rate, bolus volume, systemic blood pressure measurement, clearance measurement, or vascular access recirculation measurement.

Example 6. The medical device of any of examples 1-5, wherein the processing circuitry is further configured to determine that an indication of the parameter of interest is greater than or equal to a predetermined threshold for a predetermined period of time; and wherein the processing circuitry is configured to determine the modification to the hemodialysis treatment parameter further based on the determination that the indication of the parameter of interest is greater than or equal to the predetermined threshold for the predetermined period of time.

Example 7. The medical device of example 6, wherein the predetermined period of time is in a range of 10 minutes to 20 minutes.

Example 8. The medical device of example 6 or example 7, wherein the indication of the parameter of interest is a standard deviation of the parameter of interest from a mean of the parameter of interest.

Example 9. The medical device of example 6 or example 7, wherein the indication of the parameter of interest is a percentage of increase or decrease of the parameter of interest.

Example 10. The medical device of any of examples 1-9, further comprising: a user interface communicatively coupled to the processing circuitry, the user interface being configured to: display a list of a plurality of parameters of interest and a list of a plurality of hemodialysis treatment parameters; accept user input associating the parameter of interest with the plurality of hemodialysis treatment parameter; and wherein the processing circuitry is further configured to store the association in the memory.

Example 11. A method comprising: receiving, by processing circuitry, a signal indicative of a sensed parameter of interest from a sensor; determining, by the processing circuitry, a modification to a hemodialysis treatment parameter based on the signal indicative of the parameter of interest and an association, stored in memory, between the parameter of interest and a hemodialysis treatment parameter; and automatically modifying, by the processing circuitry, the hemodialysis treatment parameter based on the determined modification.

Example 12. The method of example 11, wherein the sensor is a first sensor, the signal is a first signal, the hemodialysis treatment parameter is a first hemodialysis treatment parameter, the association is a first association, and the modification is a first modification, further comprising: receiving, by the processing circuitry, a second signal indicative of a sensed second parameter of interest from a second sensor; determining, by the processing circuitry, a second modification to a second hemodialysis treatment parameter based on the second signal and a second association stored in memory; and automatically modifying, by the processing circuitry, the second hemodialysis treatment parameter based on the determined second modification.

Example 13. The method of example 11 or example 12, wherein the parameter of interest comprises one of blood pressure, heart rate, blood volume change, blood oxygen saturation, blood temperature, heart rate, aspiration pressure in an arterial line, inlet pressure in the arterial line, pressure in a venous line, infusion pressure, filter inlet pressure to a dialysate compartment, filter outlet pressure from the dialysate compartment, transmembrane pressure, maximum transmembrane pressure, minimum transmembrane pressure minimum, hematocrit in the arterial line, hematocrit in the venous line, hemoglobin in the arterial line, hemoglobin in the venous line, oxygen saturation in the arterial line, oxygen saturation in the venous line, glucose concentration in blood, vascular access recirculation, or Kt/V, wherein K is dialyzer clearance, t is time, and V is a volume of water in the body of a patient.

Example 14. The method of any of examples 11-13, wherein the hemodialysis treatment parameter comprises one of sodium concentration of a dialysis solution, ultrafiltration rate, total ultrafiltration volume, duration of a hemodialysis session, temperature of filtration, blood flow, dialysate flow, dialysate temperature, bicarbonate concentration of the dialysate solution, concentrate composition, total ultrafiltration volume infusion rate, bolus volume, systemic blood pressure measurement, clearance measurement, or vascular access recirculation measurement.

Example 15. The method of any of examples 11-14, further comprising: determining, by the processing circuitry, that an indication of the parameter of interest is greater than or equal to a predetermined threshold for a predetermined period of time, wherein the determining the modification to the hemodialysis treatment parameter is further based on the determination that the indication of the parameter of interest is greater than or equal to the predetermined threshold for the predetermined period of time.

Example 16. The method of example 15, wherein the predetermined period of time is in a range of 10 minutes to 20 minutes.

Example 17. The method of example 15 or example 16, wherein the indication of the parameter of interest is a standard deviation of the parameter of interest from a mean of the parameter of interest.

Example 18. The method of example 15 or example 16, wherein the indication of the parameter of interest is a percentage of increase or decrease of the parameter of interest.

Example 19. The method of any of examples 11-18, further comprising: displaying, by a user interface, a list of a plurality of parameters of interest and a list of a plurality of hemodialysis treatment parameters; accepting, by the user interface, user input associating the parameter of interest with the hemodialysis treatment parameter; and storing, by the processing circuitry, the association in the memory.

Example 20. A non-transitory computer-readable storage medium storing instructions that, when executed, cause processing circuitry to: receive a signal indicative of a sensed parameter of interest from a sensor; determine a modification to a hemodialysis treatment parameter based on the signal indicative of the parameter of interest and an association, stored in memory, between the parameter of interest and a hemodialysis treatment parameter; and automatically modify the hemodialysis treatment parameter based on the determined modification.

Various examples have been described. These and other examples are within the scope of the following claims.

What is claimed is:

1. A medical device comprising:
a sensor configured to sense a parameter of interest that changes as a function of a hemodialysis treatment parameter and generate a signal indicative of the sensed parameter of interest;
memory configured to store an association between the parameter of interest and the hemodialysis treatment parameter; and
processing circuitry configured to:
receive the signal from the sensor;
determine a modification to the hemodialysis treatment parameter based on the signal indicative of the parameter of interest and the association; and
automatically modify the hemodialysis treatment parameter based on the determined modification.

2. The medical device of claim 1, wherein the medical device is a hemodialysis device.

3. The medical device of claim 1, wherein the sensor is a first sensor, the signal is a first signal, the hemodialysis treatment parameter is a first hemodialysis treatment parameter, the association is a first association, and the modification is a first modification, the medical device further comprising:
a second sensor configured to sense a second parameter of interest that changes as a function of a second hemodialysis treatment parameter and generate a second signal indicative of the sensed second parameter of interest;
wherein the memory is further configured to store a second association between the second parameter of interest and the second hemodialysis treatment parameter, and
wherein the processing circuitry is further configured to:
receive the second signal from the second sensor;
determine a second modification to the second hemodialysis treatment parameter based on the second signal indicative of the sensed second parameter of interest and the second association; and
automatically modify the second hemodialysis treatment parameter based on the determined second modification.

4. The medical device of claim 1, wherein the parameter of interest comprises one of blood pressure, heart rate, blood volume change, blood oxygen saturation, blood temperature, heart rate, aspiration pressure in an arterial line, inlet pressure in the arterial line, pressure in a venous line, infusion pressure, filter inlet pressure to a dialysate compartment, filter outlet pressure from the dialysate compartment, transmembrane pressure, maximum transmembrane pressure, minimum transmembrane pressure minimum, hematocrit in the arterial line, hematocrit in the venous line, hemoglobin in the arterial line, hemoglobin in the venous line, oxygen saturation in the arterial line, oxygen saturation in the venous line, glucose concentration in blood, vascular access recirculation, or Kt/V,
wherein K is dialyzer clearance, t is time, and V is a volume of water in the body of a patient.

5. The medical device of claim 1, wherein the hemodialysis treatment parameter comprises one of sodium concentration of a dialysis solution, ultrafiltration rate, total ultrafiltration volume, duration of a hemodialysis session, temperature of filtration, blood flow, dialysate flow, dialysate temperature, bicarbonate concentration of the dialysate solution, concentrate composition, total ultrafiltration volume infusion rate, bolus volume, systemic blood pressure measurement, clearance measurement, or vascular access recirculation measurement.

6. The medical device of claim 1, wherein the processing circuitry is further configured to determine that an indication of the parameter of interest is greater than or equal to a predetermined threshold for a predetermined period of time; and
wherein the processing circuitry is configured to determine the modification to the hemodialysis treatment parameter further based on the determination that the indication of the parameter of interest is greater than or equal to the predetermined threshold for the predetermined period of time.

7. The medical device of claim 6, wherein the predetermined period of time is in a range of 10 minutes to 20 minutes.

8. The medical device of claim 6, wherein the indication of the parameter of interest is a standard deviation of the parameter of interest from a mean of the parameter of interest.

9. The medical device of claim 6, wherein the indication of the parameter of interest is a percentage of increase or decrease of the parameter of interest.

10. The medical device of claim 1, further comprising:
a user interface communicatively coupled to the processing circuitry, the user interface being configured to:
display a list of a plurality of parameters of interest and a list of a plurality of hemodialysis treatment parameters;
accept user input associating the parameter of interest with the plurality of hemodialysis treatment parameter; and
wherein the processing circuitry is further configured to store the association in the memory.

11. A method comprising:
receiving, by processing circuitry, a signal indicative of a sensed parameter of interest from a sensor;
determining, by the processing circuitry, a modification to a hemodialysis treatment parameter based on the signal indicative of the parameter of interest and an association, stored in memory, between the parameter of interest and a hemodialysis treatment parameter; and
automatically modifying, by the processing circuitry, the hemodialysis treatment parameter based on the determined modification.

12. The method of claim 11, wherein the sensor is a first sensor, the signal is a first signal, the hemodialysis treatment parameter is a first hemodialysis treatment parameter, the association is a first association, and the modification is a first modification, further comprising:
receiving, by the processing circuitry, a second signal indicative of a sensed second parameter of interest from a second sensor;
determining, by the processing circuitry, a second modification to a second hemodialysis treatment parameter based on the second signal and a second association stored in memory; and
automatically modifying, by the processing circuitry, the second hemodialysis treatment parameter based on the determined second modification.

13. The method of claim 11, wherein the parameter of interest comprises one of blood pressure, heart rate, blood volume change, blood oxygen saturation, blood temperature, heart rate, aspiration pressure in an arterial line, inlet pressure in the arterial line, pressure in a venous line, infusion pressure, filter inlet pressure to a dialysate compartment, filter outlet pressure from the dialysate compartment, transmembrane pressure, maximum transmembrane pressure, minimum transmembrane pressure minimum, hematocrit in the arterial line, hematocrit in the venous line, hemoglobin in the arterial line, hemoglobin in the venous line, oxygen saturation in the arterial line, oxygen saturation in the venous line, glucose concentration in blood, vascular access recirculation, or Kt/V,
wherein K is dialyzer clearance, t is time, and V is a volume of water in the body of a patient.

14. The method of claim 11, wherein the hemodialysis treatment parameter comprises one of sodium concentration of a dialysis solution, ultrafiltration rate, total ultrafiltration volume, duration of a hemodialysis session, temperature of filtration, blood flow, dialysate flow, dialysate temperature, bicarbonate concentration of the dialysate solution, concentrate composition, total ultrafiltration volume infusion rate, bolus volume, systemic blood pressure measurement, clearance measurement, or vascular access recirculation measurement.

15. The method of claim 11, further comprising:
determining, by the processing circuitry, that an indication of the parameter of interest is greater than or equal to a predetermined threshold for a predetermined period of time,
wherein the determining the modification to the hemodialysis treatment parameter is further based on the determination that the indication of the parameter of interest is greater than or equal to the predetermined threshold for the predetermined period of time.

16. The method of claim 15, wherein the predetermined period of time is in a range of 10 minutes to 20 minutes.

17. The method of claim 15, wherein the indication of the parameter of interest is a standard deviation of the parameter of interest from a mean of the parameter of interest.

18. The method of claim 15, wherein the indication of the parameter of interest is a percentage of increase or decrease of the parameter of interest.

19. The method of claim 11, further comprising:
displaying, by a user interface, a list of a plurality of parameters of interest and a list of a plurality of hemodialysis treatment parameters;
accepting, by the user interface, user input associating the parameter of interest with the hemodialysis treatment parameter; and
storing, by the processing circuitry, the association in the memory.

20. A non-transitory computer-readable storage medium storing instructions that, when executed, cause processing circuitry to:
receive a signal indicative of a sensed parameter of interest from a sensor;
determine a modification to a hemodialysis treatment parameter based on the signal indicative of the parameter of interest and an association, stored in memory, between the parameter of interest and a hemodialysis treatment parameter; and
automatically modify the hemodialysis treatment parameter based on the determined modification.
